(19) United States
(12) Patent Application Publication
(10) Pub. No.: US 2025/0261924 A1
(43) Pub. Date: Aug. 21, 2025

(54) ULTRASOUND DIAGNOSTIC TOOL FOR RENAL FIBROSIS ASSESSMENT

(71) Applicant: The Hong Kong Polytechnic University, Kowloon (HK)

(72) Inventors: Ziman CHEN, Kowloon (HK); Tin Cheung YING, Kowloon (HK)

(73) Assignee: The Hong Kong Polytechnic University, Kowloon (HK)

(21) Appl. No.: 19/051,676

(22) Filed: Feb. 12, 2025

Related U.S. Application Data
(60) Provisional application No. 63/555,741, filed on Feb. 20, 2024.

(51) Int. Cl. A61B 8/08; G16H 50/30

(52) U.S. Cl. CPC A61B 8/08; G16H 50/30

(57) ABSTRACT

A tool for using conventional ultrasound techniques for renal fibrosis assessment, particularly in cases of chronic kidney disease. Patient parameters such as age are combined with ultrasonically determined parameters both morphological (e.g. renal length) and hemodynamic (e.g. end-diastolic velocity of circulation in the interlobal renal artery) to evaluate the risk of a patient displaying moderate-to-severe renal fibrosis over a baseline risk. Additional and/or substitute parameters are also described.

### CROSS-REFERENCE TO RELATED APPLICATIONS

This application claims priority from and the benefit of U.S. Provisional Patent Application 63/555,741, filed Feb. 20, 2024, titled “Smart-CKD: A Novel Diagnostic Tool Based on Conventional Ultrasound for Renal Fibrosis Assessment in Chronic Kidney Disease”, in the United States Patent and Trademark Office. All disclosures of the document named above are incorporated herein by reference.

### BACKGROUND OF THE INVENTION

### 1. Field of the Invention

Aspects of the invention relate to a system for evaluating patients with chronic kidney disease (“CKD”) for an elevated likelihood of having moderate-to-severe renal fibrosis (“MSRF”).

### 2. Description of the Related Art

CKD is a prevalent condition affecting populations globally. The clinical manifestations of CKD include structural abnormalities in the kidneys and progressive renal dysfunction, with renal fibrosis being a common pathological pathway leading to renal failure. Timely diagnosis and accurate staging of renal fibrosis are essential for managing CKD. Currently, the “gold standard” for diagnosing and staging renal fibrosis is renal biopsy and histopathological examination. However, due to its invasive nature and associated risks, including the inability to be repeated frequently for dynamic monitoring or long-term follow-up, alternative methods for evaluating renal fibrosis are needed to improve patient care.

While the adverse impact of renal fibrosis on the clinical prognosis of CKD patients is well-established, the non-invasive and accurate identification of individuals at risk for MSRF remains a clinical challenge.

Given the impact of MSRF on CKD progression and overall health, a tool to assess the likelihood of MSRF in CKD patients would be a useful invention.

Given the importance and delicacy of the kidneys, a non-invasive tool to assess the likelihood of MSRF in CKD patients, without relying on expensive and hard-to-access diagnostic equipment like functional MRI, would be a useful invention.

Aspects of the present invention address these and other concerns.

### SUMMARY OF THE INVENTION

Aspects of the invention related to a computer-aided diagnostic tool, utilizing ultrasound (“US”) data, to discern CKD patients at an elevated likelihood of progressing towards MSRF.

This tool, referred to herein as Smart-CKD (“S-CKD,”) integrates three clinical parameters: age, ultrasonic renal length, and end-diastolic flow velocity of the interlobar renal artery. This tool provides the ability to differentiate between mild and moderate-to-severe renal fibrosis within CKD patients, thus yielding favorable clinical benefits. S-CKD can be made accessible through both an online web-based platform and an offline document-based platform, providing a user-friendly auxiliary instrument. The tool can assist healthcare practitioners in tailoring clinical decision-making and optimizing post-treatment protocols for CKD patients.

Additional aspects and/or advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is an abstract process flow diagram showing the operation of the tool.

FIG. 2 is an example ultrasonic imaging output as used in the tool specifying the evaluation procedure(s).

FIG. 3 is an abstract schematic of one complete embodiment of the tool.

### DETAILED DESCRIPTION OF THE EMBODIMENTS

Reference will now be made in detail to several embodiments of the invention that are illustrated in accompanying drawings. Whenever possible, the same or similar reference numerals are used in the drawings and the description to refer to the same or like parts or steps. The drawings are in simplified form and are not to precise scale. For purposes of convenience and clarity only, directional terms such as top, bottom, left, right, up, down, over, above, below, beneath, rear, and front, can be used with respect to the drawings. These and similar directional terms are not to be construed to limit the scope of the invention in any manner. The words attach, connect, couple, and similar terms with their inflectional morphemes do not necessarily denote direct or intermediate connections, but can also include connections through mediate elements or devices.

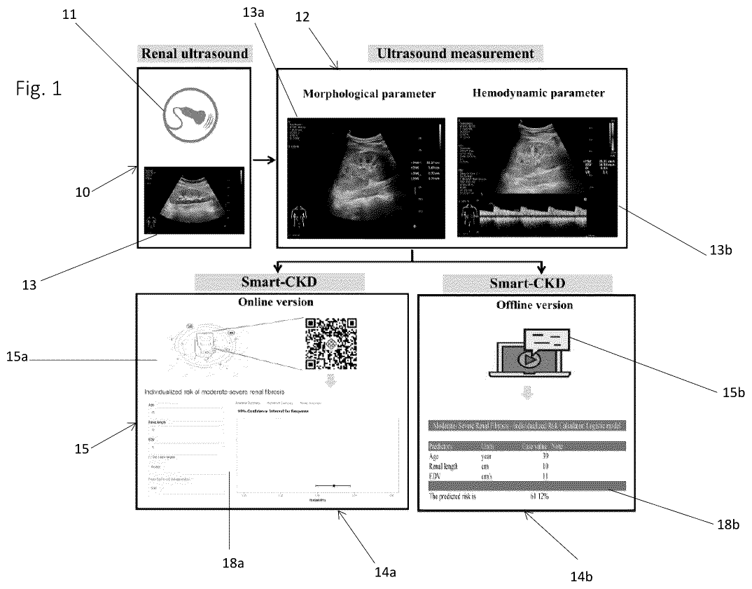

By referring to FIG. 1, the basic principle of the invention can be easily understood. A renal ultrasound system 10 uses renal ultrasound device 11 to produce renal ultrasound image data 13. Renal ultrasound image data 13 is fed into a measurement system to determine morphological parameters 13a which can include but are not limited to renal length, width, parenchyma thickness, and cortical thickness as well as renal parenchymal echogenicity. Renal ultrasound system 10 also uses Doppler measurements to measure hemodynamic parameters 13b which can include but are not limited to PSV (peak systolic velocity,) EDV (end-diastolic velocity) and RI (resistive index.)

Morphological parameters 13a and hemodynamic parameters 13b are fed into diagnostic system 15, which can comprise online system 15a and/or offline system 15b. The desired measurements are then used by the diagnostic system 15 (in either version) to calculate the probability of moderate-to-severe renal fibrosis as described further below. A health-care professional (not shown) then reviews the result of the calculation and develops an appropriate monitoring and treatment plan for the patient.

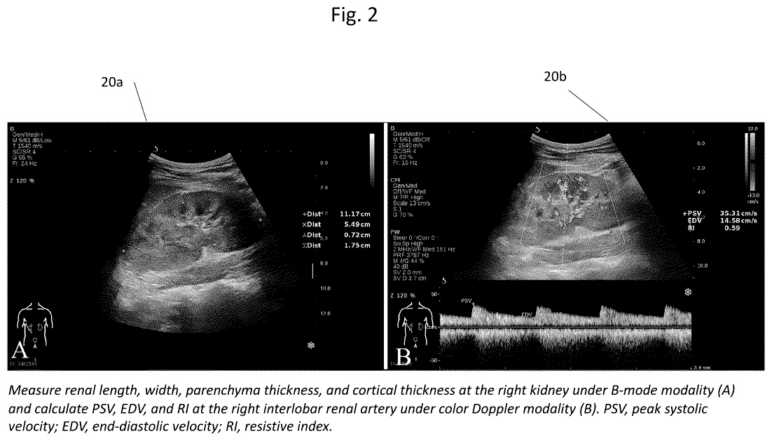

FIG. 2 shows the evaluated parameters in more detail. The tool measures renal length, width, parenchyma thickness, and cortical thickness at the right kidney under B-mode modality in morphological data 20a and calculates PSV, EDV, and RI at the right interlobar renal artery under color Doppler modality in hemodynamic data 20b.

It is known in the art that advancing age is another factor in the likelihood of development of MSRF: the patient's age is also provided to diagnostic system 15 by the operator or by an automatic record data retrieval means (not shown.)

The diagnostic system performs its calculation by using the odds-ratio formulas shown in Table 1, which indicate the risk each factor contributes over a baseline risk of developing MSRF:

TABLE 1

| Factor | Risk Increase | Odds Ratio | 95% Confidence Interval |
|---|---|---|---|
| +1 year of age | 6% | 1.06 | 1.03-1.10 |
| −1 unit of renal | 58% | 0.42 | 0.24-0.73 |
| length | | | |
| −1 unit of EDV | 20% | 0.80 | 0.68-0.94 |

By using the age of the patient, renal length, and EDV, the risk of MSRF developing in the patient being evaluated can be calculated by applying the odds-ratios shown above and performing any reasonable and appropriate statistical calculation to a baseline risk.

It is optional to substitute RI for EDV in making the calculation, as RI is proportional to EDV, but as RI also includes PSV, the calculation will lose some predictive value and thus EDV is preferred.

It is optional to use cortical thickness or parenchymal echogenicity instead of renal length for a broader evaluation of kidney structural changes over time to improve diagnostic accuracy.

It is optional to add a measurement of glomerular filtration rate (GFR) of the kidney(s) as a clinical indicator, which combines measurable functional data with the imaging data to improve diagnostic accuracy.

It is optional to integrate data from contrast-enhanced ultrasound (CEUS) for enhanced visualization of renal perfusion.

It is optional to employ elastography-based techniques for assessing tissue stiffness as an additional or substitute fibrosis indicator.

It is optional to corporate measurements of biochemical markers, such as serum creatinine or albuminuria, into the calculation to improve diagnostic accuracy. It is also optional to leverage radiomics technique(s) to extract advanced imaging biomarkers, offering additional diagnostic insights beyond visual evaluation.

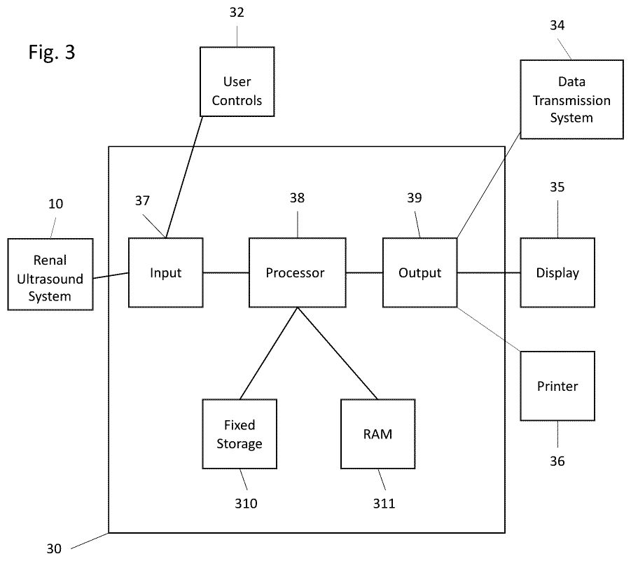

FIG. 3 shows the abstracted elements of a device implementing the invention. Device 30 is a general purpose computer, but could also be a special-purpose computer, virtual machine, or other appropriate device or collection of connected devices.

Device 30 includes input bus 37, processor 38, output bus 39, fixed storage 310, and random access memory (“RAM”) 311. Input bus 37 and output bus 39 can be unified, for example as a SCSI bus, a USB bus, or any other reasonable combination of input and output bus technology. Processor 38 can be a general-purpose CPU or IC/VLSIC, or a special-purpose CPU, VPU, or other IC/VLSIC. Most of these components are well known in the art and can comprise any desired physical component, e.g. fixed storage 310 could comprise without limitation a hard disk drive, a flash drive, one or more read-only memory chips, a virtual drive connected by a network, or any combination thereof. In place of fixed storage 310 and RAM 311, a single unified memory device such as Flash RAM can be used.

Connected to device 30 is renal ultrasound system 10, which can be connected by wire, wirelessly, or through a data bus as part of a single electronic device. When directed by a user with user controls 32, the tool performs the diagnostic calculations including any measurements not already performed by renal ultrasound system 10. Data transmission system 34 allows for a completed diagnostic calculation to be sent to a server, a remote user, or any other reasonable and appropriate recipient. Display 35 and printer 36 allow the diagnostic calculation or any other desired information to be displayed to a recipient.

To use the tool, a user initiates the tool with user controls 32. Computer instructions stored in fixed storage 310 and/or directly in RAM 311 are processed by the processor 38, which performs the evaluation of the parameters from renal ultrasound system 10 and calculation of the risk of MSRF, as described above. The results are then displayed on display 35 and/or printed on printer 36. The results can also be transmitted through data transmission system 34 to a remote display or printer. This transmission can take the form of an email, a text file, a direct transmission of a special-purpose file, or physical transfer such as on a flash drive.

A cell phone or tablet (not shown) can receive the transmission from data transmission system 34, allowing one or more health-care professionals (not shown) to singly or jointly review the result in one or more locations. The health-care professional(s) then determines whether a monitoring and/or treatment plan is appropriate.

It should be noted that while artificial intelligence and/or other large learning/machine learning methods can be used in the operation of the tool and/or the evaluation of raw datasets related to the tool's calculation methods, they are not required and form no part of the claimed invention. Ordinary multivariate logistic regression models or similar statistical tools can be applied to the measurements to allow the tool to produce diagnostic/predictive results.

Although a few embodiments of the present invention have been shown and described, it would be appreciated by those skilled in the art that changes may be made in this embodiment without departing from the principles and spirit of the invention, the scope of which is defined in the claims and their equivalents.

What is claimed is:

1. An ultrasound diagnostic tool for renal fibrosis assessment comprising:
a) a renal ultrasound system which produces renal ultrasound data related to a kidney of a patient having an age;
b) an ultrasound measurement system which evaluates the renal ultrasound data to measure at least one morphological parameter and at least one hemodynamic parameter;
c) a diagnostic system which performs at least one diagnostic calculation on the age, the morphological parameter and the hemodynamic parameter to obtain a risk assessment which predicts the patient's risk of developing moderate-to-severe renal fibrosis.

2. The tool of claim 1, wherein the patient is a person diagnosed with chronic kidney disease.

3. The tool of claim 1, wherein the morphological parameter is a renal length of the kidney.

4. The tool of claim 2, wherein the morphological parameter is a renal length of the kidney.

5. The tool of claim 1, wherein the hemodynamic parameter is an end-diastolic velocity of the interlobal renal artery.

6. The tool of claim 2, wherein the hemodynamic parameter is an end-diastolic velocity of the interlobal renal artery.

7. The tool of claim 4, wherein the hemodynamic parameter is an end-diastolic velocity of the interlobal renal artery.

8. The tool of claim 3, wherein the risk assessment uses an increased risk of 6 percent per additional year of the age in predicting the patient's risk of developing moderate-to-severe renal fibrosis.

9. The tool of claim 3, wherein the risk assessment uses an increased risk of 58 percent for every one unit decrease in the renal length in predicting the patient's risk of developing moderate-to-severe renal fibrosis.

10. The tool of claim 9, wherein the risk assessment uses an increased risk of 58 percent for every one unit decrease in the renal length in predicting the patient's risk of developing moderate-to-severe renal fibrosis.

11. The tool of claim 5, wherein the risk assessment uses an increased risk of 20 percent for every one unit decrease in the end-diastolic velocity in predicting the patient's risk of developing moderate-to-severe renal fibrosis.

12. The tool of claim 10, wherein the risk assessment uses an increased risk of 20 percent for every one unit decrease in the end-diastolic velocity in predicting the patient's risk of developing moderate-to-severe renal fibrosis.

13. The tool of claim 12, wherein the patient is a person diagnosed with chronic kidney disease.

14. An ultrasound diagnostic tool for renal fibrosis assessment, comprising a processor coupled to a memory, a fixed storage system, a renal ultrasound system, and an output bus, wherein the fixed storage system and/or the memory is configured to store a computer code, and the processor is configured to execute the computer code for:
performing a series of renal ultrasound measurements on a patient to obtain a set of renal ultrasound data;
evaluating the renal ultrasound data to obtain a set of morphological parameters and a set of hemodynamic parameters;
performing at least one diagnostic calculation on the set of morphological parameters and the set of hemodynamic parameters to obtain a risk assessment which evaluates the patient's risk of developing moderate-to-severe renal fibrosis;
outputting the risk assessment through the output bus to a display and/or a printer and/or a remote device which can display the risk assessment to a health-care professional.

15. The tool of claim 14, wherein the morphological parameter is a renal length of the kidney.

16. The tool of claim 15, wherein the hemodynamic parameter is an end-diastolic velocity of the interlobal renal artery.

17. The tool of claim 16, wherein the risk assessment uses an increased risk of 6 percent per additional year of the age in predicting the patient's risk of developing moderate-to-severe renal fibrosis.

18. The tool of claim 17, wherein the risk assessment uses an increased risk of 58 percent for every one unit decrease in the renal length in predicting the patient's risk of developing moderate-to-severe renal fibrosis.

19. The tool of claim 18, wherein the risk assessment uses an increased risk of 20 percent for every one unit decrease in the end-diastolic velocity in predicting the patient's risk of developing moderate-to-severe renal fibrosis.

20. The tool of claim 19, wherein the patient is a person diagnosed with chronic kidney disease.
